(19) United States
(12) Patent Application Publication
(10) Pub. No.: US 2019/0293646 A1
(43) Pub. Date: Sep. 26, 2019

(54) Method for rapid and direct identification of microbial pathogen from positive culture sterile body fluids using mass spectrometry

(71) Applicant: Liping Feng, Baltimore, MD (US)

(72) Inventor: Liping Feng, Baltimore, MD (US)

(21) Appl. No.: 15/933,809

(22) Filed: Mar. 23, 2018

(51) Int. Cl. G01N 33/569; G01N 33/68; C12Q 1/24

(52) U.S. Cl. CPC G01N 33/56911; G01N 33/56961; C12Q 1/24; G01N 33/6848

(57) ABSTRACT

The invention provides methods for rapid isolation of microorganisms from positive culture sterile body fluids, including blood, cerebrospinal fluid (CSF), pleural fluid, ascitic fluid, pericardial effusion, joint cavity fluid, vitreous fluid, and amniotic fluid for mass spectrometry identification. Whenever the subject of blood culture is discussed, the intended sample used is always related to blood sample. However, it is also necessary to be aware that other than the blood sample, sterile fluids can also be inoculated as samples for blood culture testing. Among the sterile fluids that are commonly known are CSF, pleural fluid, ascitic fluid, pericardial effusion, joint cavity fluid, vitreous fluid, amniotic fluid etc. The methods involve combining micro-volume positive blood culture sample with detergent solution to lyse human blood cells, then isolating the microorganism by differential centrifugation process that first removes interfering substances such as charcoal (when present), resins and human blood cellular debris through a low speed centrifugation, then isolates the microorganisms in the sample supernatant through a fast centrifugation. The methods not only apply to regular blood culture media but also apply to antimicrobial removal containing media such as resin containing BD BACTEC™ Plus-Aerobic media and charcoal-containing Biomerieux BacT/Alert® FA media. In addition, the methods can isolate a variety of Gram-positive bacteria, Gram-negative bacteria, and yeast in clinical settings. The isolated microorganism(s) from positive blood culture can be used for multiple downstream analyses, including identification of the microorganism(s) by mass spectrometry, phonotypical, or molecular identification methods.

### BACKGROUND OF THE INVENTION

The presence of microorganism recovered from sterile fluids is potentially life-threatening specifically in cases of sepsis, meningitis, pericarditis, peritonitis, septic arthritis and empyema. Therefore, it is of paramount importance to identify such microorganisms so that early treatment can be initiated. Sepsis is one of the leading causes of death of both adults and children worldwide, imposing a heavy human and economic burden in both the developed and developing world. About 20 million cases of severe sepsis arise every year globally. In the US, septicemia places consistently in the top 10 causes of death, with mortality of 25%-50%. Sepsis-related mortality in the United States is greater than that of stroke, especially in the face of emerging resistance of causative organisms. Rapid identification of causative pathogen for sepsis is considered crucial for optimal management of these infections. Blood cultures are still considered to be ‘gold standard’ for the detection of microbial pathogens related to bacteremia and sepsis. Blood culture systems perform continuous monitoring of bacterial growth in blood culture vessels in semi-automated incubators. When a blood culture bottle flags positive as growing bacteria, Gram's stain allows for preliminary identification, while definite identification and susceptibility testing by phenotypic and genotypic methods are routinely performed with solid-medium subcultures. Thus, final results often arrive more than 72 hours after sampling. The outcomes for septic patients have been shown to be dependent on the adequacy of early antimicrobial chemotherapy. Timely antibiotic administration in patients with sepsis is critical, with each hour of delay (over the first 6 hours) resulting in a 7.6% decrease in survival. As definitive identification of the causative organism through traditional culture methods often requires 24-72 hours, this delay can lead to administration of either inadequate or overly broad antimicrobial therapy. Inappropriate antimicrobial selection may result in therapy-related complications, emergence of antimicrobial resistance, and increases in patient morbidity, mortality, and costs. Therefore, direct identification of pathogens and antimicrobial susceptibility testing directly from positive blood culture is an ideal solution. Matrix-Assisted Laser Desorption Ionisation-Time of Flight (MALDI TOF) Mass

Spectrometry is widely recognized as a rapid and accurate identification system for microorganisms based on whole cell analysis. This technique is extremely simple and quick: bacteria is directly sampled from an agar plate or broth (or a simple extract can be made) onto a MALDI-TOF plate, overlaid with a matrix, fired with a laser, and the resulting spectra of proteins (mainly ribosomal proteins) are recorded and compared against reference spectra in the database to provide identification, with confidence to species or genera level. Here, we describe a new method for direct rapid identification of microbial pathogens from positive blood culture sample using mass spectrometry. Our method detects microbial pathogens within 30 minutes and with high accuracy rate compared to the current microbial identification systems such as BD Phoenix system (BD diagnostics, Sparks, USA) and the Biomerieux Vitek system (BioMérieux, Marcy l'Étoile, France), which requires 18-48 hours. Therefore, the described method shorten the traditional pathogen isolation and identification process from positive blood culture from 18-48 hours to just 30 minutes, a change that would significantly improve patient care.

### BRIEF SUMMARY OF THE INVENTION

The invention is based on the method with which microbial pathogens can be recovered from micro-volume positive blood culture samples, regardless of the type of blood culture bottles. There are two major types of commercial blood culture bottles on the market—the Bactec system (BD Diagnostics, Sparks, USA) and the BacT/Alert system (Biomerieux, France). The methods described herein apply to all types of blood culture bottles, including the resin-containing BD BACTEC™ Plus-Aerobic bottle and the charcoal-containing BacT/Alert® FA bottle. To date, methods for direct identification of microbial pathogens from blood culture are limited. The current commercial Sepsityper kit (Bruker Daltonik GmbH, Bremen, Germany) is recommended and optimized for blood culture bottles without any charcoal supplements, because these supplements negatively affect subsequent identification by mass spectrometry. Clinical studies have already shown that the Sepsityper kit has difficulties with the BacT/Alert system bottles, particularly with blood culture bottles containing charcoal, so in clinical settings, the Sepsityper kit should be avoided for rapid identification from charcoal-containing blood cultures. In addition, Sepsityper does not work well for Gram-positive microorganism from positive blood culture. Therefore, there is a need to develop a universal method to meet the clinical needs for direct identification of microbial pathogens from all types of positive blood culture media bottles. The methods described meet the clinical needs and also perform well for both Gram-negative and Gram-positive bacterial identification from all types of positive blood culture for mass spectrometry identification.

The methods include lysing human blood cells in micro-volume positive blood culture samples with lysis buffer containing detergent, then isolating the microbial pathogens by a differential centrifugation process, which removes the cellular debris and charcoal that would normally interfere with mass spectrometry analysis of cellular proteins. The differential centrifugation process begins by centrifuging the culture sample at low speed to remove cellular debris and charcoal, then pelleting bacterial cells at high speed, followed by washing the pelleted microorganism for accurate mass spectrometry identification.

In one embodiment, a method for isolating bacterial cells from a micro-volume positive blood sample includes: i) obtaining a positive blood culture sample that contains at least one Gram-positive or Gram-negative bacteria from the charcoal-containing BacT/Alert® FA bottle; ii) mixing the sample with a detergent solution to lyse human blood cells for short time incubation; iii) removing the human blood cellular debris and charcoal by centrifugation at low speed first and transferring the supernatant to a new micro centrifuge tube; iv) pelleting bacterial cells by centrifugation the supernatant at high speed in a new micro centrifuge tube. The pelleted bacterial cells by the differential centrifugation process, which is followed by washing with DI water, are used to perform mass spectrometry analysis for identification by comparing the spectrum of the given isolated microorganism with the reference mass spectrum of an isolate from the database that comes with the instrument.

In another embodiment, the methods described herein can be used to isolate yeast from a micro-volume positive sample. This method for isolating yeast cells from a positive BD BACTEC™ Plus-Aerobic media includes: i) obtaining a positive blood culture sample that contains at least one species of yeast from BD BACTEC™ Plus-Aerobic media bottle; ii) mixing the sample with a detergent solution to lyse human blood cells for short time incubation; iii) removing the human blood cellular debris and charcoal by centrifugation at low speed first and the transferring the supernatant to a new centrifuge tube; iv) pelleting yeast cells by centrifuging the supernatant at high speed. The pelleted yeast cells by the differential centrifugation process, which is followed by washing with DI water, are used to perform mass spectrometry analysis for identification by comparing the spectrum of the given isolated microorganism with the reference mass spectrum of an isolate from the database that comes with the instrument.

### RIEF DESCRIPTION OF THE DRAWINGS

FIG. 1 illustrates the method of one embodiment described herein for isolating Gram-positive or Gram-negative bacteria from a positive blood culture sample with differential centrifugation steps for mass spectrometry identification.

FIG. 2 illustrates the method of one embodiment described herein for isolating yeast cells from a positive blood culture sample with differential centrifugation steps for mass spectrometry identification.

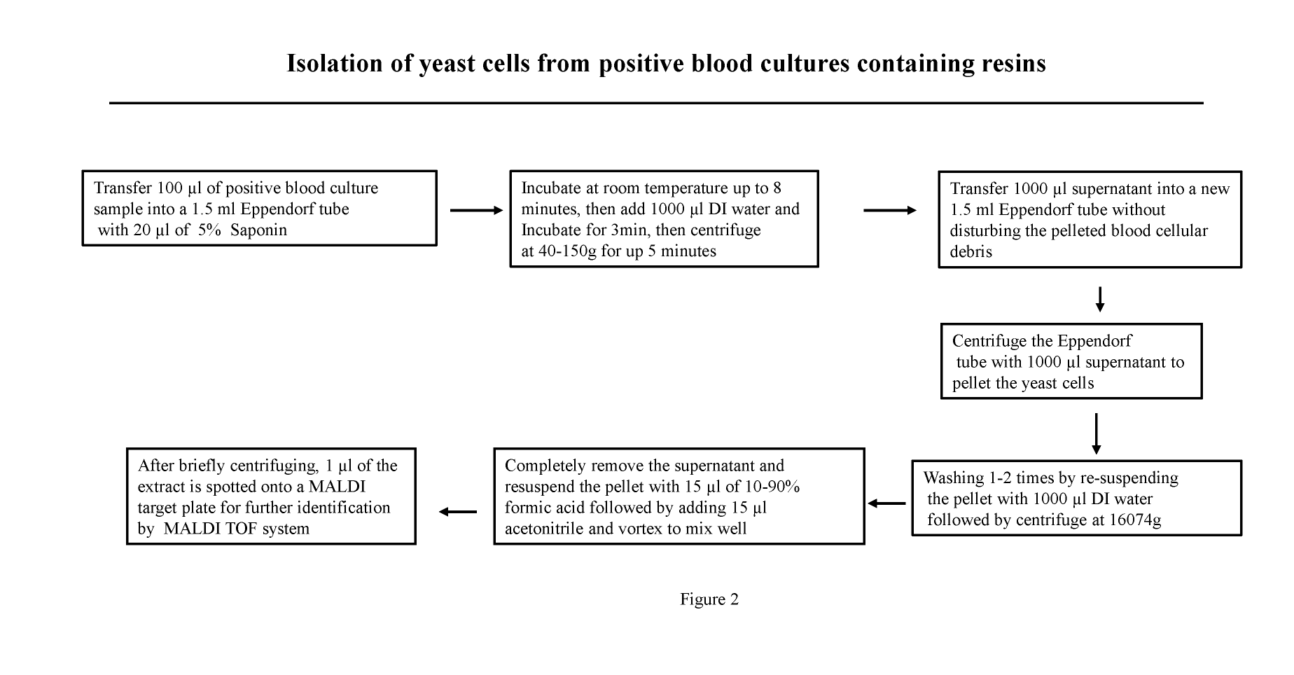

### DETAILED DESCRIPTION

Embodiments described herein provide methods for isolating a wide range of microorganisms from a positive blood culture media for subsequent mass spectrometry identification.

In one embodiment, a method for recovering bacterial pathogens from charcoal-containing BacT/Alert® FA bottles recovering at least one microorganism from a patient for mass spectrometry identification.

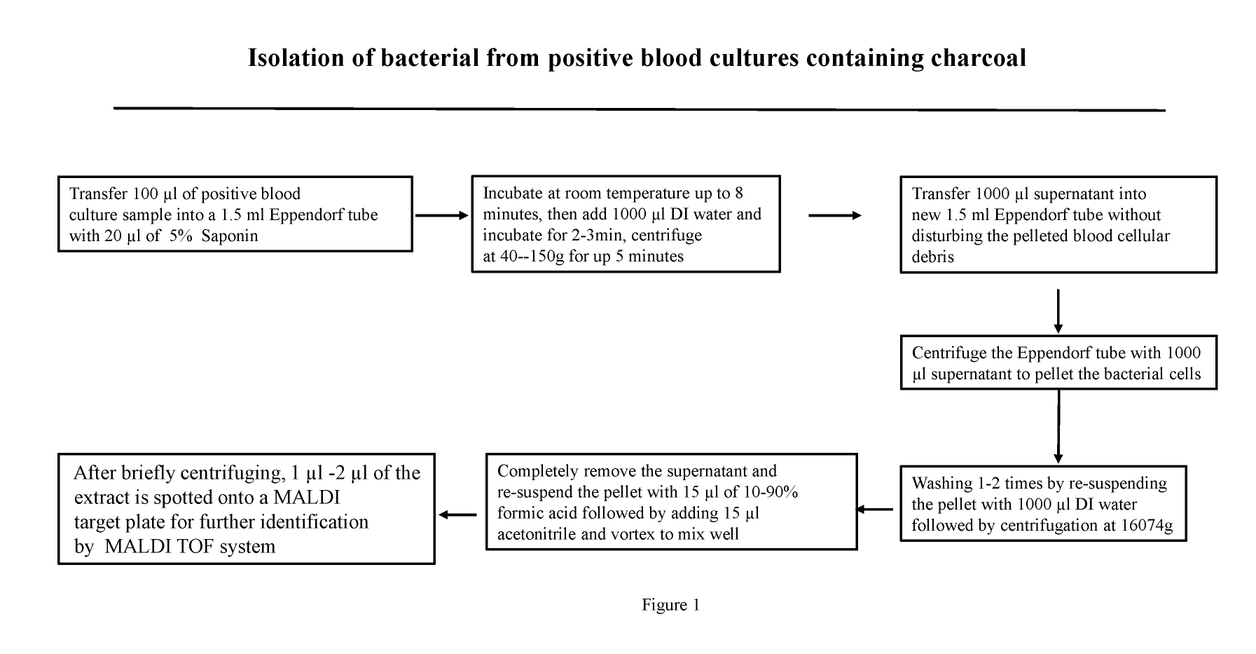

FIG. 1 illustrates one method of the various embodiments described herein. 100 μl of positive blood culture sample obtained from a positive blood culture bottle containing charcoal is mixed with 20 μl of 5% saponin in a 1.5 ml Eppendorf tube, and the mixed sample is incubated at 35° C. for up to 8 min to completely lyse the human blood cells. Then, 1000 μl DI water is added to the mixed sample to incubate at 35° C. for 3 min, and is followed by centrifugation at 700 rpm (46.6×g) for 1 minute to remove charcoal particles and human blood cellular debris, while the supernatant containing the Gram-positive or Gram-negative bacteria is carefully transferred to a new 1.5 ml Eppendorf tube and is centrifuged at 13000 rpm (16074×g) to pellet the bacterial cells. After the supernatant is carefully removed, the pellet containing the bacterial cells is rinsed twice by resuspending the pellet with 1000 μl DI water followed by centrifugation at 13000 rpm (16074×g) to pellet the bacterial cells. The supernatant is then completely removed, and the isolated bacterial cells in the sample tube are briefly centrifuged at 13000 rpm (16074×g) and are lysed with 15 μl formic acid and 15 μl of acetonitrile to release the soluble ribosome proteins, 1-2 μl of the supernatant containing the ribosome proteins is spotted onto MALDI TOF sample support plate and is allowed to air dry. The dried sample is overlaid with 1 μl of MALDI matrix (HCCA) solution (prepared by dissolving 2.5 mg of HCCA in 250 μl of 2.5% trifluoroacetic and 47.5% acetonitrile in DI water) and is allowed to air dry again, then is analysed by mass spectrometry. This sample preparation method applies to all of the available commercial blood culture culturing media such as BacT/ALERT blood culture media (bioMérieux, Durham, N.C.), the Bactec blood culture media (BD Microbiology, Cockeysville, Md.) etc. In one embodiment, the method provides the procedure to isolate Gram positive or Gram negative bacteria from BacTAlert charcoal-containing blood culture bottles; the absorbent charcoal is used to remove antimicrobials in the blood for the direct identification of microorganisms by MALDI TOF mass spectrometry. In another embodiment, the method applies to the positive Bactec blood culture media bottle containing resins, which is also for the removal of antibiotics in the blood, for subsequent MALDI TOF mass spectrometry analysis. The method also can be used for the isolation of yeast from positive blood culture bottles.

The detergent used to lyse human blood cells in the embodies is not limited to saponin. Any detergent or in combination with other type of detergents that can completely lyse human blood cells without destroying the microorganisms will be accepted. The final concentration of the detergent used for the method is not limited to the concentration used in the embodies described herein.

The isolated microorganisms can be used for identification with mass spectrometry, manual or automated AST testing, and different molecular or phenotypic identification testing. They also can be used for resistance marker analysis to provide timely therapeutic options to guide the management of infectious diseases resulting from resistant bacterial infections.

### EXAMPLES

### Example 1

Isolation of Bacterial Strains from Positive Aerobic BacT/ALERT FA Charcoal Blood Culture Bottle for Mass Spectrometry Identification

TABLE 1
Microbial identification by MALDI-TOF versus Classical ID/VITEK2
from Biomerieus FAN ® media with activated charcoal

| | Number of | | |
|---|---|---|---|
| | isolates | Correct ID | Correct ID |
| Microorganism | analyzed | VITEK2 | MALDI TOF |
| Acinetobacter baumannii | 2 | 2 | 2 |
| Enterococcus faecium | 1 | 1 | 1 |
| Enterococcus faecalis | 1 | 1 | 1 |
| Escherichia coli | 2 | 2 | 2 |
| Enterobacter cloacae | 3 | 3 | 3 |
| Klebsiella pneumoniae | 4 | 4 | 4 |
| Staphylococcus epidermidis | 3 | 3 | 3 |
| Staphylococcus aureus | 4 | 4 | 4 |
| Staphylococcus warneri | 2 | 2 | 2 |
| Streptococcus pneumoniae | 2 | 2 | 0 |
| Stenotrophomonas maltophilia | 2 | 2 | 2 |
| Total | 26 | 26 | 24 |

A total of 26 positive aerobic BacT/ALERT FA bottles from our routine diagnostic laboratory were consecutively identified by VITEK 2 with a statistical probability >99% according to the manufacturer's instructions and analyzed by direct mass spectrometry. The samples were prepared with the method below. Results of direct MAI DI-TOF MS from positive BacT/ALERT bottles and VITEK 2 are shown in Table I.

For the MALDI TOF sample preparation, 150 μl of each sample was combined with 30 μl of 5% saponin in an Eppendorf tube, and the mixed sample was incubated at 35° C. for up to 8 minutes to lyse the human blood cells. Then, 1000 μl of DI water was added into the sample tube to completely lyse the blood cells by incubating at 35° C. for 3 minutes, followed by centrifugation at 700 rpm (46.6×g) for 1 minute to remove the charcoal particles and visible and invisible human blood cellular debris, while the supernatant containing the Gram-positive or Gram-negative bacteria was carefully transferred to a new 1.5 ml Eppendorf tube for centrifugation at 13000 rpm (16074×g) to pellet the bacterial cells. After the supernatant was carefully removed, the pellet cells were rinsed by re-suspending with 1 ml DI water, followed by centrifugation at 13000 rpm (16074×g) to pellet the bacterial cells. The supernatant was completely removed, and the isolated bacterial cells in the sample tube were lysed with 15 μl formic acid and 15 μl of acetonitrile to release soluble ribosome proteins, after briefly centrifugation at 13000 rpm (16074×g) for 1 min. Finally, 1-2 μl of the supernatant containing the ribosome proteins was spotted onto MALDI TOF sample support plate for identification by mass spectrometry.

The results show that method described here was able to correctly identify 24/26 of the organisms from clinically positive blood culture samples compared to the VITEK2 system, the latter which was based on conventional biochemical identification that is currently found in most microbiology laboratories. The results indicate that MALDI TOF offered higher identification accuracy rate at the species level when compared to VITEK 2. Additionally, MALDI-TOF dramatically shortens the identification time from 24-36 hours to just 30 minutes.

### Example 2

Isolation of Bacterial Pathogens from Positive BD BACTEC™ Plus-Aerobic Bottle for Mass Spectrometry Identification

A total of 28 positive BD BACTEC™ Plus(resin)-Aerobic bottles from our routine diagnostic laboratory were consecutively identified by VITEK 2 with a statistical probability ≥99% according to the manufacturer's instructions and analyzed by direct mass spectrometry. The samples were prepared with the method below. Results of direct MALDI-TOF MS from positive BD BACTEC™ Plus (with resin)-Aerobic bottles and \TREK 2 are shown in Table 2.

For the MAT DI TOF sample preparation, 100 μl of each sample was combined with 20 μl of 5% saponin in an Eppendorf tube, and the mixed sample was incubated at room temperature for up to 8 minutes to lyse the human blood cells. Then 1000 μl of DI water was added into the tube to completely lyse the blood cells by incubating at room temperature for 3 minutes, followed by centrifugation at 700 rpm (46.6×g) for 1 minute to remove the charcoal particles and visible and invisible human blood cellular debris, while the supernatant containing the Gram-positive or Gram-negative bacteria was carefully transferred to a new 1.5 ml Eppendorf tube for centrifugation at 13000 rpm (16074×g) to pellet the bacterial cells. After the supernatant was carefully removed, the pellet was rinsed by re-suspending with 1 ml DI water followed by centrifugation at 13000 rpm (16074μ) to pellet the bacterial cells. The supernatant was completely removed, and the isolated bacterial cells in the sample tube was lysed with 15 μl formic acid and 15 μl of acetonitrile to release soluble ribosome proteins, after brief centrifugation at 13000 rpm for 1 min. Finally, 1-2 μl of the supernatant containing the ribosome proteins was spotted onto MALDI TOF sample support plate for identification by mass spectrometry.

For comparison, the sample was also prepared using a prior art method (U.S. Pat. No. 8,569,010B2), namely, 900 μl of positive blood culture sample was added into 1 ml centrifuge tube, 200 μl lysis buffer was added into the sample buffer and the sample was mixed by vortexing for approximately 10 seconds, followed by centrifugation for 2 minutes at 10,000 g, the supernatant was removed completely and the pellet containing isolated microorganisms was re-suspended in 1 ml of DI water and

TABLE 2
Microbial identification by MALDI-TOF versus Classical ID/VITEK2
from BD BACTEC ™ Plus(resin)-Aerobic media

| | | | Correct ID | |
|---|---|---|---|---|
| | Number of | Correct | MALDI TOF | |
| | isolates | ID | New | Prior Art |
| Microorganism | analyzed | VITEK | Method | method |
| Enterococcus faecalis | 2 | 2 | 2 | 0 |
| Enterococcus faecium | 4 | 4 | 4 | 0 |
| Staphylococcus epidermidis | 6 | 6 | 6 | 3 |
| Staphylococcus hominis | 2 | 2 | 2 | 2 |
| Staphylococcus aureus | 7 | 7 | 6 | 2 |
| Streptococcus mitis | 1 | 1 | 1 | 0 |
| Streptococcus pneumoniae | 2 | 2 | 1 | 0 |
| Streptococcus anginosus | 1 | 1 | 1 | 0 |
| Staphylococcus warneri | 2 | 2 | 2 | 0 |
| Total | 28 | 28 | 25(89%) | 5(25%) |

centrifuged for 2 min at 10,000 g. The supernatant was removed completely, then 300 μl of DI water was added to the tube and vortexed thoroughly, followed by adding 900 μl of ethanol in the tube and mixing again. The mixture was centrifuged at 10,000 g for 2 minutes, the ethanol was removed completely, then 50 μl of formic acid was added to the pellet containing microorganism and the mixture was vortexed thoroughly followed by adding 50 μl of acetonitrile into the tube. The mixture of the sample was mixed by vortexing and centrifuged again at 10,000 g for 1 min. 1-2 μl of the supernatant was spotted onto a MALDI TOF sample support plate and allowed it to air dry, the dried sample was overlaid with 1 μl of MALDI matrix (HCCA) solution (prepared by dissolving 2.5 mg of HCCA in 250 μl of 2.5% trifluoroacetic and 47.5% acetonitrile in DI water) and allowed the sample to air dry, followed by analysis by mass spectrometry. All mass spectrometry data was recorded on a Clin-TOF II MS with a BioExplorer V4.3 software.

The prior art method resulted in 6/28 (21%) correct identification for 14 bacterial pathogens from blood stream infection patients, while the methods described herein correctly identified 14/14(100%) pathogens from the same patients. The results indicate that the methods described herein in the invention can be used to isolate and identify bacterial pathogens from positive blood culture of sepsis pathogens, including Gram-positive, Gram-negative bacteria, and yeast.

Although the invention herein has been described with reference to particular embodiments, it is to be understood that these embodiments are merely illustrative of the principles and applications described herein. It is therefore to be understood that numerous modifications may be made to the illustrative embodiments and that other arrangements may be devised without departing from the spirit and scope of the various embodiments described herein as defined by the appended claims.

1. A method of isolating microorganism from positive culture sterile body fluids, including blood, cerebrospinal fluid (CSF), pleural fluid, ascitic fluid, pericardial effusion, joint cavity fluid, vitreous fluid, and amniotic fluid for microbial identification by mass spectrometry, the method comprising:
Obtaining a positive blood culture sample from blood culture media determined to contain microorganism(s);
Combining the sample with human blood cell lysis reagent and incubating the mixed sample in a centrifuge tube;
Isolating the microorganism in the prepared sample by differential centrifugation;

2. The method of claim 1, wherein the positive blood culture sample is from blood culture bottles containing or not containing an antimicrobial removal substance that is charcoal, resins or another antimicrobial removal substance;

3. The method of claim 1, wherein the microorganism can be Gram-positive bacteria, Gram-negative bacteria, and/or yeast.

4. The method of claim 1, wherein the lysis buffer contains at least one detergent selected from a group consisting of sodium dodecyl sulfate (SDS), sopanin, triton X-100, or a combination thereof;

5. The method of claim 4, wherein the concentrations of detergent used is appropriate for completely lysing blood cells but keep the bacterial cells intact to be identified by mass spectrometry identification.

6. The method of claim 1, wherein the differential centrifugation process comprising:
Centrifuging the sample combined with lysis buffer at a low speed to remove charcoal, resins, visible and invisible cellular debris and retain the microorganism in the supernatant;
Transferring the supernatant to a new microcentrifuge tube to pellet the microorganism at a fast speed followed by washing the pelleted microorganism;

7. The method of claim 6, wherein the washing pelleted microorganism comprising:
Washing the pelleted microorganism by resuspending the with DI water followed by centrifugation at fast speed;
Optionally, washing the pelleted microorganism 1-2 more times.

8. The method of claim 7 comprising:
Treating the pelleted microorganism by appropriate volume of formic acid after completely removing the liquid portion followed by adding the same volume of acetonitrile;
Centrifuging the micro-centrifuge sample tube at fast speed;
Spotting 1-2 μl of supernatant onto MALDI TOF sample support plate after centrifuging the sample tube at fast speed for further mass spectrometry identification.
